(19) United States
(12) Patent Application Publication
(10) Pub. No.: US 2012/0078199 A1
(43) Pub. Date: Mar. 29, 2012

(54) TEMPERATURE GAUGE FOR ENEMA BAG

(76) Inventor: Charlotte Shirley, Richmond, OH (US)

(21) Appl. No.: 12/889,442

(22) Filed: Sep. 24, 2010

(51) Int. Cl. A61M 3/02

(52) U.S. Cl. 604/262

(57) ABSTRACT

An apparatus to administer an enema comprising: an enema bag, where enema bag includes a fill top opening; a thermostat attached to one side of the enema bag; and a tubing, where said tubing is inserted into a patient's rectum. The thermostat may provide either Celsius or Fahrenheit temperature readings of the fluid within the enema bag.

### BACKGROUND OF THE INVENTION

1. Field of Invention

The present invention relates to an enema bag that includes a temperature gauge to read and monitor fluid temperature within the bag.

2. Description of Related Art

An enema is a procedure that introduces liquids into the rectum and colon of an individual. The fluids are introduced via the anus and the purpose is principally for medical reasons such as constipation or an encopresis. Enemas are also used as rehydration therapy for patients who are incapable of receiving I.V. therapy. The main medical uses of enemas include bowel stimulation, relief of constipation, cleaning of the lower bowel prior to a surgical procedure, administration of medications into the rectum and injection of contrast substances for radiological imaging of the bowel. Certain individuals also use enemas as an alternative medicine or detoxification treatment. Further some enemas are used for recreational use as related to sexual activities.

With respect to medical administration of enemas, it is necessary to monitor the temperature of the water or fluid within the enema bag when administering a medical enema. The fluids within the bag should match the natural body temperature of the individual to prevent internal scalding or patient discomfort when the fluids are introduced into the rectum. Enema bags are traditionally used for enemas are incapable of temperature monitoring and therefore it's difficult for a professional to determine and regulate the appropriate temperature of the fluids within the enema bag. It would therefore be advantageous to have a temperature gauge directly on an enema bag that helps the medical professional regulate and determine the appropriate temperature of the fluids within the bag.

### SUMMARY OF THE INVENTION

The present invention relates to an apparatus to administer an enema comprising: an enema bag, where enema bag includes a fill top opening; a thermostat attached to one side of the enema bag; and a tubing, where said tubing is inserted into a patient's rectum. The thermostat may provide either Celsius or Fahrenheit temperature readings of the fluid within the enema bag.

### BRIEF DESCRIPTION OF DRAWINGS

FIG. 1 depicts a prospective view of an enema bag with a temperature gauge according to the present invention.

### DETAILED DESCRIPTION

The present invention relates to an enema bag that includes a temperature gauge to provide an indication of the temperature of the fluids within the bag during an enema procedure. The enema bag according to the present invention includes a thermostat attached thereto in order to monitor the temperature of the fluids within the enema bag. The use of such a temperature monitoring procedure helps to ensure that the administrator is aware of the temperature of the fluids within the enema bag. The thermostat insures that the temperature of the fluids is close to or matches the body temperature of the patient.

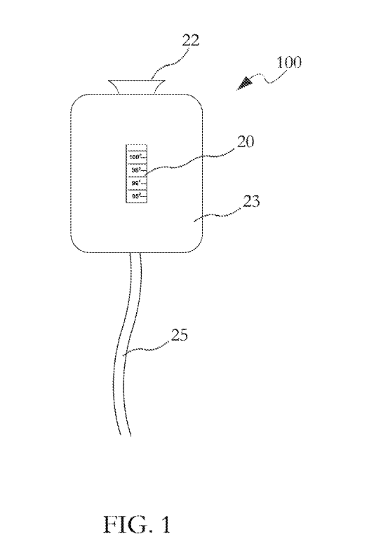

In reference to FIG. 1, a temperature gauge enema bag 100 is depicted. The enema bag 100 includes a temperature gauge 20 affixed to one side. The temperature gauge 20 is attached to the one side 23 of the enema bag 100. The enema bag 100 further includes a fill top 22 that provides a means for the insertion of fluid within the enema bag and tubing 25, which extends outwardly from the opposite side of the enema bag 100. The tubing 25 is inserted into a patient's rectum to administer the enema. As fluid is inserted into the enema bag 100, the temperature gauge 20 provides a means to accurately detect the temperature of the fluids within the enema bag. The application of the temperature gauge enables the administrator or clinical worker to determine and regulate the fluid being administered during the enema.

The temperature gauge 20 can provide temperature readings in either Fahrenheit or Celsius as needed or desired. Further the temperature gauge is attached to the enema bag in a manner to readily sense the temperature of the fluids within the enema bag. The sensing time is essentially instantaneous; therefore the user may have a quick reading of the temperature of the fluids within the enema bag. Use of this temperature gauge therefore enables the user to effectively measure the temperature of the fluids and to ensure that the fluids being administered are in an acceptable range to prevent any injury or discomfort to the patient.

What is claimed is:

1. An apparatus to administer an enema comprising:
a. an enema bag, where enema bag includes a fill top opening;
b. a thermostat attached to one side of the enema bag; and
c. a tubing, where said tubing is inserted into a patient's rectum.

2. The apparatus according to claim 1, where said thermostat provides at least one of Celsius and Fahrenheit readings of the fluid within the enema bag.
